(19) United States
(12) Patent Application Publication
(10) Pub. No.: US 2024/0358936 A1
(43) Pub. Date: Oct. 31, 2024

(54) ATOMIZING NOZZLE AND NASAL DRUG DELIVERY DEVICE

(71) Applicant: WUXI NEST BIOTECHNOLOGY CO., LTD, WUXI (CN)

(72) Inventors: Feng CHEN, WUXI (CN); Weidong YANG, WUXI (CN)

(73) Assignee: WUXI NEST BIOTECHNOLOGY CO., LTD, WUXI (CN)

(21) Appl. No.: 18/770,650

(22) Filed: Jul. 12, 2024

Related U.S. Application Data
(63) Continuation of application No. PCT/CN2023/095258, filed on May 19, 2023.

(30) Foreign Application Priority Data
Feb. 6, 2023 (CN) 202320153814.3

(51) Int. Cl. A61M 11/00

(52) U.S. Cl. CPC A61M 11/007; A61M 2210/0618

(57) ABSTRACT

The present disclosure discloses an atomizing nozzle and a nasal drug delivery device. The atomizing nozzle includes a spray hood which includes a conical hood body and a connecting cylinder provided inside the hood body and connected to the inner top of the hood body, the wall of the connecting cylinder is provided with a bayonet structure or a claw structure for non-detachable snap fit with the corresponding claw structure or the bayonet structure on the outer side of the syringe extending into the connecting cylinder, a columnar end extends from the external tip of the hood body for extending into the internal nasal cavity, the connecting cylinder, the columnar end and a portion of the hood body therebetween jointly form a communicating channel, the front end of the columnar end is provided with a spray hole, and a fluid-guiding cylinder.

### FIELD

The present disclosure relates to the field of medical devices, and specifically relates to an atomizing nozzle, and a nasal drug delivery device.

### BACKGROUND

Nasal drug delivery is a new and promising method of drug delivery. In clinical medicine, it has been used to some extent, for example, spraying medication into the nasal cavity to treat nasal diseases such as rhinitis. Nasal drug delivery has the advantages of convenient operation, no pain, and the drug can be directly applied to the lesion, etc. It will be more widely used in the future in the treatment of infants and young children, respiratory diseases, and vaccination. At present, nasal spray new crown vaccine, nasal spray influenza vaccine have been marketed, the subsequent nasal spray will have a wider range of applications.

The common atomized spray nozzle for nasal drug delivery is mounted on the bottle, and the user relies on his/her senses to extend the nozzle into the nasal cavity to deliver the drug. If the medication is to be administered to a child or other person who is unable to accurately grasp the depth of the nozzle into the nasal cavity, there is a risk that this nasal medication delivery device will easily reach too far into the nasal cavity, which could result in damage to the nasal cavity. In addition, the atomizing nozzle is usually reused many times until the medication in the medication bottle is used up, but using the same atomizing nozzle many times will cause the atomizing nozzle to be contaminated, and it is easy to bring some bacteria, viruses and the like into the nasal cavity during the process of atomizing the medication.

In order to solve the above problems existing in the existing technology, there is an urgent need for a nasal drug delivery technology in this field, which can realize the effect of one-time use of the nasal drug delivery device, and intervene in the depth of the spray nozzle of the nasal drug delivery device extending into the nasal cavity, to avoid the spray nozzle extending into the nasal cavity too deep, causing damage to the nasal mucosa, and improve the safety of the drug delivery process and operation of the nasal drug delivery device.

### SUMMARY

As the key technology of modern trains, train network control technology can be used to ensure the effectiveness, safety and comfort of train control. It is indispensable for heavy freight trains, high-speed passenger trains, or urban rail transit vehicles.

In order to overcome the above defects existing in the prior art, the present disclosure provides an atomizing nozzle, and a nasal drug delivery device, capable of realizing the effect of one-time use of the nasal drug delivery device, and interfering with the depth of the nozzle of the nasal drug delivery device reaching into the nasal cavity, to avoid that the nozzle reaches into the nasal cavity at a distance that is too deep, causing damage to the nasal mucosa, and to enhance the safety of the drug delivery process and operation of the nasal drug delivery device.

In one embodiment, the above-described atomizing spray nozzle provided according to the embodiments of the present disclosure comprises: a spray hood, comprises a conical hood body and a connecting cylinder provided inside the hood body and connected to the inner top of the hood body, the wall of the connecting cylinder being provided with a bayonet structure or a claw structure for non-detachable snap fit with the corresponding claw structure or the bayonet structure on the outer side of the syringe extending into the connecting cylinder, a columnar end extending from the external tip of the hood body for extending into the internal nasal cavity, the connecting cylinder, the columnar end and the hood portion in between them jointly form a communicating channel, the front end of the columnar end being provided with a spray hole, and

a fluid-guiding cylinder, provided in the communicating channel and having an interference fit with the inner wall of the channel, the upper end of the fluid-guiding cylinder being provided with a liquid outflow hole, the fluid in the fluid-guiding cylinder flowing out through the liquid outflow hole and being guided to be sprayed out from the spray hole.

In some embodiments, the bayonet structure or the claw structure on the wall of the connecting cylinder is located on the lower side of the wall of the connecting cylinder below the bottom horizontal plane of the hood body.

In some embodiments, the inner lower side edge of the connecting cylinder is provided with at least one locating portion recessed in a direction close to the hood body, the locating portion being in the same longitudinal axis as the bayonet structure or the claw structure on the lower side of the barrel wall of the connecting cylinder to circumferentially locate the bayonet structure or the claw structure on the outer side of syringe extending into the connecting cylinder.

In some embodiments, the cylinder is curved, and tangents are made along two arc endpoints of the curved ends of the lower edge, and the angle between the tangents and the horizontal plane ranges from 0° to 70°.

In some embodiments, the angle between tangent and horizontal plane is 18°.

In some embodiments, the angle between the conical surface of hood body and the central axis ranges from 20° to 60°.

In some embodiments, the extension of columnar end ranges from 1 to 6 mm and its diameter ranges from 4 to 8 mm.

In some embodiments, the upper end of the fluid-guiding cylinder is provided with at least one radially open liquid outflow hole, and the interior of the spray hood is provided with at least one layer of fluid atomization structure composed of fan-shaped projections, the fan shaped bulges are evenly distributed along the circumference of the spray holes, and to pressurize the liquid flowing out through the liquid outflow hole to guide it to spiral forward to the spray hole.

In some embodiments, the fluid atomization structure further comprises a conical channel, the conical channel tapering in cross-section at an end proximate to the spray hole to pressurize the fluid flowing to the spray hole.

In addition, the above-described nasal drug delivery device provided according to the embodiment of the present disclosure comprises the atomizing nozzle provided in the above-described embodiments; and a syringe, the upper end of the syringe being inserted into the lower end of the atomizing nozzle and snap-fit with the atomizing nozzle.

### BRIEF DESCRIPTION OF THE DRAWINGS

The above embodiments of the present disclosure will be better understood after reading the detailed description of the embodiments of the present disclosure in conjunction with the following figures. In the figures, components are not necessarily drawn to scale, and components having similar related features may have the same or similar reference numerals.

FIG. 1A shows a schematic diagram of the structure of a nasal drug delivery device provided according to some embodiments of the present disclosure;

FIG. 1B shows a sectional view of the nasal drug delivery device shown in FIG. 1A;

FIG. 2 shows a front view of a spray hood in an atomizing nozzle according to some embodiments of the present disclosure;

FIG. 3A is a sectional view of the spray hood in the atomizing nozzle shown in FIG. 2.

FIG. 3B is a sectional view of the fluid guide cylinder in the atomizing nozzle shown in FIG. 2.

FIG. 4 shows a sectional view of the spray hole according to some embodiments of the present disclosure; and

FIG. 5 shows a sectional view of the syringe provided according to some embodiments of the present disclosure.

### REFERENCE SIGNS

100 nasal drug delivery device
200 atomizing nozzle
210 spray hood
211 hood body
212 connecting cylinder
213 columnar end
2130 chamfer
214 spray hole
2140 fluid atomization structure
2141 fan-shaped projection
2142 conical channel
215 bayonet structure
2150 lower edge
216 positioning part
220 fluid-guiding cylinder
221 liquid outflow hole
300 syringe
310 claw structure
320 needle barrel
321 outlet
330 push-rod
331 piston
3310 installation body
3311 extension part
340 needle
341 needle cap

### DETAILED DESCRIPTION OF EMBODIMENTS

The embodiments of the present disclosure are described in the following detailed description. Although the description of the present disclosure will be described in conjunction with the embodiments, this is not a limitation of the present disclosure. On the contrary, the disclosure is described in connection with the embodiments to cover other alternatives or modifications that are possible in the embodiments of the present disclosure. In order to provide a thorough understanding of the present disclosure, many specific details are included in the following description. The present disclosure may also be practiced without these details. In addition, some specific details are omitted in the description in order to avoid confusing or obscuring the present disclosure.

In the description of the present disclosure, it should be noted that the terms “installation”, “connecting”, and “connected” should be understood broadly unless explicitly stated and defined otherwise. For example, the terms “installation”, “connecting”, and “connected” may be either a fixed connection, a detachable connection, or an integral connection; the terms may be either a mechanical connection or an electrical connection; the terms also may be either a direct connection, an indirect connection through an intermediate medium, or an internal connection between two components. The specific meaning of the above terms in the present disclosure can be understood in the art.

In addition, “up”, “down”, “left”, “right”, “top”, “bottom”, “horizontal”, “vertical” used in the following description shall be understood as the orientation described in the paragraph and shown in the related figure. The relative term is used for convenience of description only, and does not mean that the device described therein is to be manufactured or operated in the specific orientation, and therefore should not be construed as limiting the present disclosure.

Understandably, although the terms “first”, “second”, “third”, etc. may be used to describe various components, regions, layers and/or portions to distinguish different components, regions, layers and/or portions, the order of these components, regions, layers and/or portions described above should not be limited by the terms. Therefore, a first component, region, layer and/or portion mentioned below may be also mentioned as a second component, region, layer and/or portion without departing from some embodiments of the present disclosure.

As described above, the common atomized spray nozzle for nasal drug delivery is mounted on the bottle, and the user relies on his/her senses to extend the nozzle into the nasal cavity to deliver the drug. If the medication is to be administered to a child or other person who is unable to accurately grasp the depth of the nozzle into the nasal cavity, there is a risk that this nasal medication delivery device will easily reach too far into the nasal cavity, which could result in damage to the nasal cavity. In addition, the atomizing nozzle is usually reused many times until the medication in the medication bottle is used up, but using the same atomizing nozzle many times will cause the atomizing nozzle to be contaminated, and it is easy to bring some bacteria, viruses and the like into the nasal cavity during the process of atomizing the medication.

In order to solve the above problems in the prior art, the present disclosure provides an atomizing nozzle and a nasal drug delivery device, which can realize the effect of one-time use of the nasal drug delivery device, and intervene in the depth of the spray nozzle of the nasal drug delivery device extending into the nasal cavity, to avoid the spray nozzle extending into the nasal cavity too deep, causing damage to the nasal mucosa, and improve the safety of the drug delivery process and operation of the nasal drug delivery device.

In some non-limiting embodiments, the above-described atomizing nozzle provided by the embodiment of the present disclosure may be configured in the above-described nasal drug delivery device provided by the embodiments of the present disclosure.

The operation of the above-described atomizing nozzle will be described below in connection with some embodiments of the nasal drug delivery device. The embodiments of the nasal drug delivery device are only some non-limiting embodiments provided herein to clearly demonstrate the main idea of the present disclosure and to provide some specific solutions that can be easily implemented by the public, and are not intended to limit the whole way of working or the whole function of the nebulizing nozzle. Similarly, the atomizing nozzle is only one non-limiting embodiment provided in this case and does not constitute a limitation on these embodiments of the subject nasal drug delivery device.

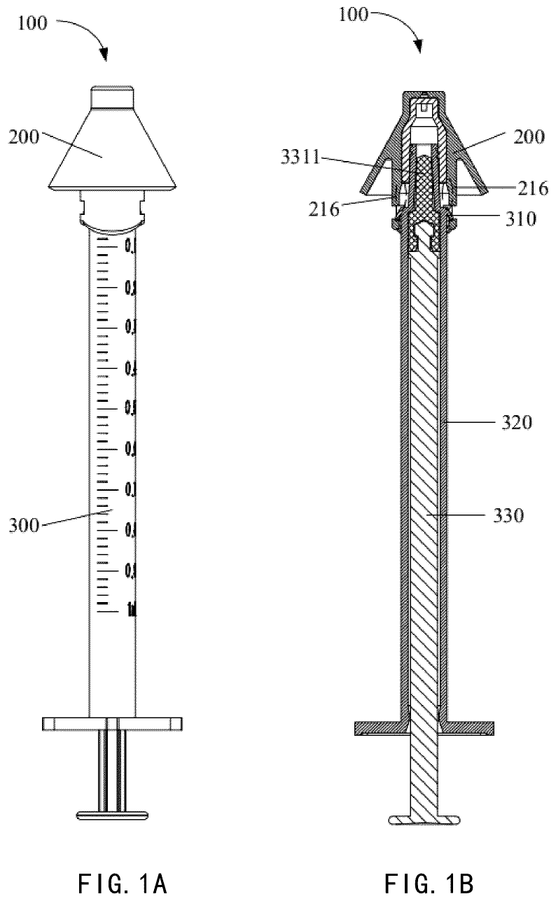

Please refer to FIGS. 1A and 1B. FIG. 1A shows the schematic structure of a nasal drug delivery device provided according to some embodiments of the present disclosure. FIG. 1B is a sectional view of the nasal drug delivery device shown in FIG. 1A.

In some non-restrictive embodiments of the present disclosure, as shown in FIGS. 1A and 1B, the nasal drug delivery device 100 mainly comprises an atomizing nozzle 200 and a syringe 300.

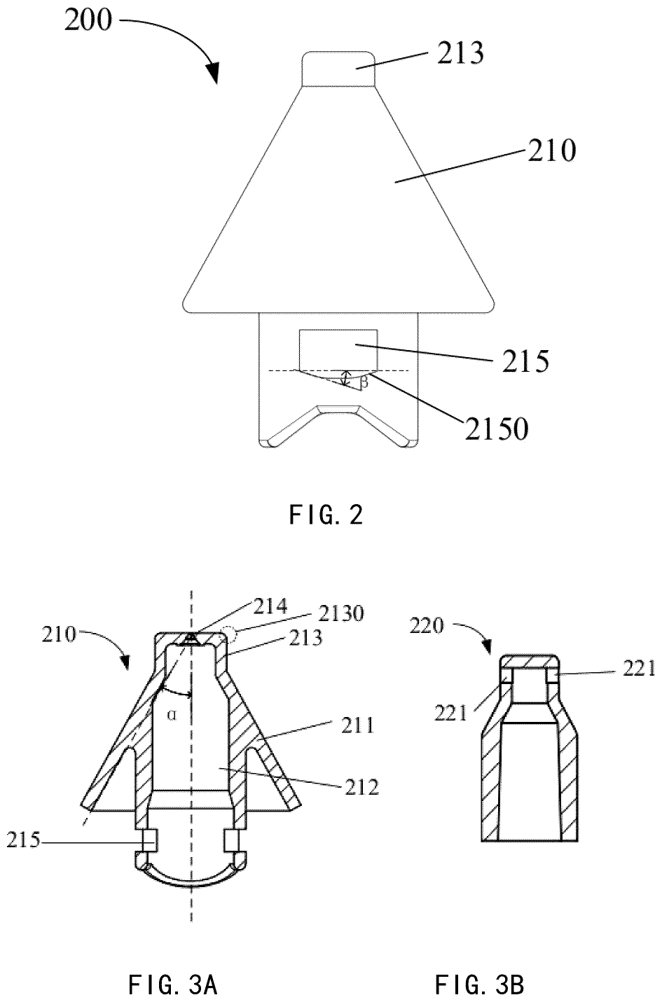

In one embodiment, please refer to FIG. 2 and FIGS. 3A and 3B. FIG. 2 shows a front view of a spray hood in an atomizing nozzle according to some embodiments of the present disclosure. The atomizing nozzle 200 mainly comprises a spray hood 210 and a fluid-guiding cylinder 220.

FIG. 3A is the sectional view of the spray hood in the atomizing nozzle shown in FIG. 2 above, and FIG. 3B is the sectional view of the fluid-guiding cylinder in the atomizing nozzle shown in FIG. 2. As shown in FIGS. 3A and 3B, the fluid-guiding cylinder 220 is arranged inside the spray hood 210. The spray hood 210 includes a conical hood body 211 and a connecting cylinder 212 arranged inside the hood body 211 and communicated with the inner tip of the hood body 211.

A section of columnar end 213 is protruded from the outer tip of the conical hood body 211 at the end of the smaller cross-section of the hood body 211, and a spray hole 214 is provided at the front end of the columnar end 213 for the outflow of the liquid agent. In one embodiment, the cross section of the columnar end 213 at the top of the hood body 211 can be designed to fit the shape of the inside of the human nasal cavity in a round or similar shape, that is, the columnar end 213 has a cylindrical structure as a whole, and it is not only easy to reach into the nasal cavity of the drug subject for spray delivery, but also has the function of centring and can be aligned with the internal passage of the nasal cavity for drug injection.

In one embodiment, as shown in FIG. 3A, in some embodiments, a chamfer 2130 may also be provided at the front edge of columnar end 213, and a rounded edge chamfer 2130 may further avoid damage to the nasal mucosa caused by columnar end 213 during extension.

In some embodiments, the length range of the columnar end 213 may be set from 1 to 6 mm, and its diameter range may be set from 4 to 8 mm. In some embodiments of the present disclosure, for some special populations, such as children, the length of the columnar end 213 may be to be 3 mm and the diameter may be to be 6 mm, and the column end 213 can be inserted into the child's nasal cavity appropriately without causing the child's nasal discomfort.

The above scheme, which is adapted to the size of the child's nasal passage, at the end of the column 213, is only a non-restrictive mode of implementation offered in this disclosure, intended to clearly demonstrate the main idea of the disclosure and to provide a concrete scheme that is easy for the public to implement, and not to limit the scope of protection in this disclosure. The size of the columnar end 213 can also be designed to fit inside the nasal cavity of adults and other specific populations.

In this disclosure, the hood body 211 is arranged in a conical shape, and the outer surface of its lower end, which is spread out like an umbrella, is in contact with the opening of the nasal cavity, which can prevent the columnar end 213 of the atomizing nozzle 200 from being excessively inserted into the inside of the nasal cavity, to cause damage to the nasal mucosa.

In some embodiments, the range of angle α between the conical surface of the hood body 211 and its central axis, i.e. the angle α of one half of the conical apex angle, may be set in the range of 20° to 60°. Further, the angle α of half of the top angle of the cone can also be set up according to the age of the user. For example, the hood body 211 can be set for infants, children, adults, etc., and the hood body used by infants has a smaller angle, and the hood body used by adults has a relatively larger angle α.

Continuing to FIG. 3A, in some embodiments of the present disclosure, the connecting cylinder 212 is a cylinder structure connected at both ends, the upper end of which is connected to the inner wall of the hood body 211. The inner wall of the connecting cylinder 212, the inner wall of the top of the hood body 211 and the inner part of the columnar end 213 jointly define the communicating channel, and the end of the communicating channel is connected with the spray hole 214.

In some embodiments, the lower end of the hood body 211 may be a cantilever structure, and the upper end of the connecting cylinder 212 is in the cantilever structure and connected to the inner wall of the hood body 211. In some other embodiments, the hood body 211, the connecting cylinder 212, and the columnar end 213 may also be designed as a single forming structure, or separate bodies and assembled by means of pressing or medical adhesives.

Next, in conjunction with FIGS. 1B and 3B, in some embodiments a fluid-guiding cylinder 220 is provided inside the connecting cylinder 212 of the spray hood 210. The fluid-guiding cylinder 220 can be pressed into the communicating channel from the lower port of the connecting cylinder 212. The upper end of the fluid-guiding cylinder 220 is provided with a liquid outflow hole 221 with at least one radial opening. The liquid outflow hole 221 is connected with a spray hole 214 in the connecting cylinder 212 to guide the liquid agent in the fluid-guiding cylinder 220 to spray out of the spray hole 214 via the liquid outflow hole 221 through the communicating channel.

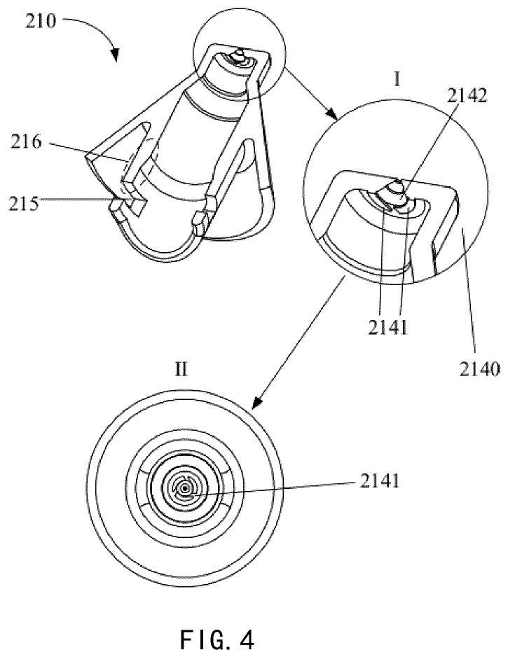

Further, refer to FIG. 4, FIG. 4 shows a sectional view of the spray hole according to some embodiments of the present disclosure.

The fluid-guiding cylinder 220 is pressed into the spray hood 210, and the upper end of the fluid-guiding cylinder 220 may be an anvil surface that touches the interior of the spray hood 210. In one embodiment, as shown in partial enlargements I and II in FIG. 4, the anvil face of the fluid-guiding cylinder 220 is in contact with fan-shaped projections 2141 inside the spray hood 210. At least one layer of fluid atomization structure 2140 is provided at the front end of the inside of the spray hood 210 near the spray hole 214, which is composed of multiple pieces similar to the fan-shaped projection 2141. The fan-shaped projections 2141 are uniformly arranged along the circumference of spray hole 214 and the liquid agent exiting through the liquid outflow hole 221 can be pressurized to spiral forward to spray hole 214. For example, in some optional embodiments, as shown in partial enlargement II, three fan-shaped projections 2141 can be arranged, and the three projections are uniformly arranged in a circumferential direction.

According to FIG. 3B and FIG. 4, the upper side wall of the fluid-guiding cylinder 220 is provided with two liquid outflow holes 221 having radial opening, through which the liquid agent in the syringe 300 is discharged and flows to the fluid atomization structure 2140. When the liquid agent flows from the liquid outflow hole 221 to the spray hole 214, the liquid agent forms a spiral liquid flow in the fluid atomization structure 2140, and there is a large pressure drop when the liquid agent is ejects from the spray hole 214.

Further, as shown in locally enlarged figures I and II of FIG. 3, the fluid atomization structure 2140 may also include a conical channel 2142 located between the multiple fan-shaped projections 2141 and the spray hole 214. The conical channel gradually narrows in cross section at one end near the spray hole 214. To guide the liquid agent through the gradually reduced cross-sectional area of the conical channel to continue to pressurize spiral forward, the pressure suddenly drops at the spray hole 214 to achieve the spray effect of atomization.

In other embodiments, the conical channel 2142 may also be provided in the form of steps to have the same effect of directing the flow of the fluid and increasing the fluid pressure, as shown in the locally enlarged FIG. 1.

Next, please continue to combine FIG. 1B and FIG. 3A, the lower side of the connection cylinder 212 can be provided with a clamp structure suitable for the fixed fit of needle barrel 320 of syringe 300. In one embodiment, the upper end of syringe 300 is inserted into the connecting cylinder 212, and in some embodiments, as shown in FIG. 2, the wall of the connecting cylinder 212 may be provided with a bayonet structure 215 to correspond to the claw structure 313 provided on the outer wall of the upper end of the syringe 300. During the process of inserting syringe 300 into connecting cylinder 212, the claw structure 310 on the outer wall is deformed elastic, and the deformation of the claw structure 310 is restored after the claw structure 310 enters the bayonet structure 215 of connecting cylinder 212. The bayonet structure 215 on the connecting cylinder 212 and the claw structure 310 on the syringe 300 correspond to each other to form a non-removable one-time clamping fit, and the connecting cylinder 212 and the syringe 300 cannot be disassembled without causing damage to the clamping structure.

Due to the small total dose required for vaccination and the long interval between vaccinations, it is no longer suitable for the delivery device of the prior art with the atomizer mounted on the vial. The liquid agent in syringe 300 in this disclosure is usually a one-time dose, so there is no need to pump the agent into syringe 300 after injection, realizing the one-time use of nasal drug delivery device 100, avoiding the contamination of atomizing nozzle 200 caused by multiple use of nasal drug delivery device 100 in the prior art. As a result, some bacteria and viruses are brought into the nasal cavity during the process of atomizing drug delivery.

The non-detachable one-time clamping scheme consisting of the bayonet structure 215 on the connecting cylinder 212 and the clamping claw structure 310 on the syringe 300 provided above is only a non-restrictive mode of implementation provided in the present disclosure, intended to clearly demonstrate the main idea of the present disclosure and to provide a specific scheme for public implementation, but not to limit the scope of protection in this disclosure. In some other embodiments, a claw structure may be arranged on the wall of the connecting cylinder 212, and a corresponding bayonet structure may be arranged on the outer wall of the upper end of the syringe 300 for a one-time clamping fit with each other.

In some embodiments, as shown in FIG. 3, the bayonet structure 215 (or gripe structure) on the wall of the lower end of the connecting cylinder 212 may be positioned outside the cantilevering hood body 211, i.e., the bayonet structure 215 on the wall of the connecting cylinder 212 or the claw structure is located below the bottom horizontal plane of the hood body 211. Thus, it is convenient for the user to directly observe the position of the bayonet structure 215 (or the claw structure), and the connecting cylinder 212 and the needle barrel 320 of the syringe 300 are quickly and accurately stuck together.

Further, in conjunction with FIGS. 1B and 4, in some embodiments, the inner lower edge of the connecting cylinder 212 has a positioning part 216 recessed in the direction close to the hood body 211, and the bayonet structure 215 of the connecting cylinder 212 is on the same longitudinal axis with the positioning part 216. During the one-time clamping assembly of the syringe 300 and the connecting cylinder 212 of the atomizing nozzle 200, the upper end of syringe 300 can be inserted into the atomizing nozzle 200, and the clamping position can be performed by rotating syringe 300 (that is, rotating the central axis of syringe 300 as the axis).

In one embodiment, when the external claw structure 310 of the barrel 320 of syringe 300 is rotated to the positioning part 216, it is just aligned with the position of bayonet structure 215, and the user can perceive that it is difficult to continue to rotate the syringe 300, and then the syringe 300 can be pressed in the direction close to the atomizing nozzle 200. The claw structure 310 on the syringe 300 is clamped into the bayonet structure 215 of the connecting cylinder 212. The setting of the positioning part 216 above makes the clamping operation more convenient and faster, and saves the assembly time.

In some embodiments, as shown in FIG. 1B, the inner lower side edge of the connecting cylinder 212 may be provided with two symmetrical positioning parts 216 extending in a direction away from the hood body 211, forming a butterfly edge. Accordingly, the outer wall of the needle barrel 320 of the syringe 300 is also provided with two symmetrical claw structures 310. By setting two pairs or even more pairs of non-detachable one-time fitting bayonet structure 215 and claw structure 310, and each pair of bayonet structure 215 and claw structure 310 can be aligned with each other, and then complete one-time pressing and clamping, further improving the installation convenience.

Further, as shown in FIG. 2, the lower edge 2150 of the bayonet structure 215 on the lower side of the wall of the connecting cylinder 212 is curved downward, and the endpoints of both ends of the arc of the lower edge 2150 are tangent. The angle β between the tangent line and the horizontal plane can range from 0° to 70°. The lower edge 2150 of the bayonet structure 215 is arranged into an upward opening with a downward arc shape where the two ends are higher than the lowest point in the middle. After the bayonet structure 215 is connected with the clamping claw structure 310, the atomizing nozzle 200 is twisted to make the clamping claw structure 310 move to the end of the lower edge 2150 of the bayonet structure 215. The end of the syringe 300 is pushed deeper into the atomizer 200, and the outlet 321 on the upper end of the syringe 300 fits more closely with the fluid-guiding cylinder 220.

In one embodiment, the angle range between the tangent line and the horizontal plane can be further set in the range of 15° ˜25° in an embodiment of the present disclosure, taking into account the effort of twisting the atomizing nozzle 200 with respect to the syringe 300. The angle β between the arc tangent line of the lower edge 2150 of the bayonet structure 215 on the lower side of the wall of the connecting cylinder 212 and the horizontal plane can be set to 18°, and the upper outlet 321 of the syringe 300 and the fluid-guiding cylinder 220 can be fitted more closely after rotation.

In some embodiments, the fluid-guiding cylinder 220 is pressed into the spray hood 210 from the lower end of the connection cylinder 212, and the fluid-guiding cylinder 220 and the connection cylinder 212 are assembled together by interference force to form a primary seal structure. Further, the outlet 321 at the front end of the needle barrel 320 of syringe 300 is inserted into the fluid-guiding cylinder 220, and a secondary sealing structure can be formed between the inner wall of the fluid-guiding cylinder 220 and the outer wall of the outlet 321 of barrel 320. The setting of the primary seal structure and the secondary seal structure can avoid the possible leakage risk between the syringe 300 and the atomizing nozzle 200.

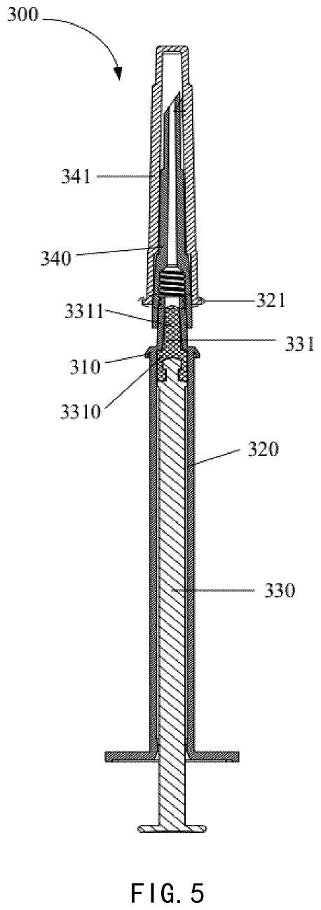

Refer next to FIG. 5, which shows a sectioned view of the syringe provided according to some embodiments of the disclosure.

As shown in FIG. 5, the structure of syringe 300 in the nasal drug delivery device 100 mainly comprises a needle barrel 320 and a push-rod 330 inserted into the needle barrel 320. The front end of the push-rod 330 is equipped with a rubber piston 331. The outer wall of the needle barrel 320 is provided with a claw structure 310, and the outer side of the claw structure 310 is a free end, which can make the claw structure 310 elastic deformation. In the process of clamping the claw structure 310 on the outer upper end of the barrel 320 with the bayonet structure 215 on the lower end of the connecting cylinder 212 in the atomizing nozzle 200, the claw structure 310 deformation occurs. After entering the bayonet structure 215, the claw structure 310 then recovers the deformation, not to damage the claw structure. The claw structure 310 is difficult to exit from the bayonet structure 215, realizing the effect that the nasal drug delivery device 100 can only be used once, avoiding the risk of infection caused by the contamination of the nasal drug delivery device 100 after reuses, and the clamp structure of the bayonet and the claw itself is simple in structure and convenient to manufacture.

Further, the size of syringe 300 can be selected according to the dose size. The size of outlet 321 of syringe 300 of different specifications can be set to the same size and they can be assembled with atomizer 200. As shown in FIG. 5, the nasal delivery device 100 can also be configured with a disposable needle 340 and a needle cap 341 in order to facilitate the suction of the liquid agent into the barrel 320 of the syringe 300. When the nasal drug delivery device 100 is used, a disposable needle 340 can be first installed on the outlet 321 of barrel 320. The needle 340 is inserted into the vial for drug absorption, and the drug is transferred to the barrel 320. Then, the disposable needle 340 is removed, and the atomizing nozzle 200 is assembled to the outlet 321 of the barrel 320 for nasal administration. After the dosing is completed, the atomizing nozzle 200 cannot be removed for a second inhalation, so the effect of one-time use is achieved. Further, going back to FIG. 1A, syringe 300 can also be equipped with a dose marking scale on barrel 320, and the specification of syringe 300 can be selected according to the dosage, thus reducing the waste of medicine. Compared with the prior art, the dosage of nasal drug delivery device 100 provided in this disclosure is more accurate. The dosage of the drug can be controlled more precisely.

In one embodiment, a dose stopper may also be mounted on the push-rod 330 of the syringe 300 (not shown in the attached drawing). The dose stopper can be disassembled for dose control and accurate dosing of the nasal cavity. In one embodiment, the dose in syringe 300 is the amount of drug used in both nasal cavities, the dose stopper can be mounted on the push-rod 330, and the push-rod 330 can be pushed until it cannot move, and the pushed dose is the dose given to one of the nasal cavities. Subsequently, the dose stopper is removed, at which point the push-rod 330 can continue to be administered intranasally to the other side. The dose limiter is an existing structure and will not be described here.

In some embodiments, the front end of the outlet 321 of barrel 320 may be threaded to fit with the thread on needle 340 to avoid disconnection between needle 340 and barrel 320 during drug transfer. Further, the thread may be arranged at the very front end of the outlet 321 and the rear end of the outlet 321 may have sufficient length to form the secondary seal structure between the outlet 321 and the fluid-guiding cylinder 220.

In some embodiments of the present disclosure, as shown in FIG. 5, the rubber piston 331 in syringe 300 has an installation body 3310 and an extension part 3311, and the extension part 3311 is located at the upper end of the installation body 3310 and is integrated with the installation body 3310. The lower end of the installation body 3310 is provided with an opening for the upper end of the push-rod 330 to be inserted. The diameter of the installation body 3310 is equivalent to the diameter of the barrel 320, and a sealing structure can be formed between them.

As shown in FIG. 1B, the cross-sectional area of the extension part 3311 is smaller than that of the installation body 3310, and the extension part 3311 can be smoothly inserted into the fluid-guiding cylinder 220. When the extension part 3311 is inserted into the fluid guide tube 220, it takes up most of the space in the fluid guide tube 220, to discharge as much of the agent in the fluid guide tube 220 as possible, reducing the dead volume in the single-use nasal drug delivery device 100. For example, in some embodiments, the dead volume within the nasal delivery device 100 is reduced to 0.053 ml, which is a relatively small amount of dead volume, to reduce the waste of the agent. In some better embodiments, the amount of dead volume can be further reduced by lengthening the extension part 3311 to 0.05 ml, 0.04 ml, etc.

In summary, this disclosure provides an atomizing nozzle and a nasal drug delivery device, which can realize the one-time use effect of the nasal drug delivery device, and interfere with the depth of the nozzle of the nasal drug delivery device into the nasal cavity, to avoid too deep distance of the nozzle into the nasal cavity, causing damage to the nasal mucosa, and improve the safety of the drug delivery process and operation of the nasal drug delivery device.

1. An atomizing nozzle, comprising:
a spray hood, comprising a conical hood body and a connecting cylinder provided inside the hood body and connected to an inner top of the hood body, the wall of the connecting cylinder is provided with a bayonet structure or a claw structure for non-detachable snap fit with a corresponding claw structure or a bayonet structure on an outer side of a syringe extending into the connecting cylinder, a columnar end extends from an external tip of the hood body for extending into an internal nasal cavity, the connecting cylinder, the columnar end and a portion of the hood body there between jointly form a communicating channel, a front end of the columnar end is provided with a spray hole, and
a fluid-guiding cylinder, provided in the communicating channel and having an interference fit with an inner wall of the channel, an upper end of the fluid-guiding cylinder is provided with a liquid outflow hole, the fluid in the fluid-guiding cylinder flows out through the liquid outflow hole and is guided to be sprayed out from the spray hole.

2. The atomizing nozzle of claim 1, wherein the bayonet structure or the claw structure on the wall of the connecting cylinder is located on a lower side of the wall of the connecting cylinder below a bottom horizontal plane of the hood body.

3. The atomizing nozzle of claim 1, wherein an inner lower side edge of the connecting cylinder is provided with at least one locating portion recessed in a direction close to the hood body, the locating portion is in the same longitudinal axis as the bayonet structure or the claw structure on the lower side of the wall of the connecting cylinder to circumferentially locate the bayonet structure or the claw structure on the outer side of syringe extending into the connecting cylinder.

4. The atomizing nozzle of claim 3, wherein a lower edge of the bayonet structure on the lower side of the wall of the connecting cylinder is curved, and tangents are made along two arc endpoints of the lower edge, and the angle between the tangents and the horizontal plane ranges from 0° to 70°.

5. The atomizing nozzle of claim 4, wherein the angle between tangent and horizontal plane is 18°.

6. The atomizing nozzle of claim 1, wherein the angle between a conical surface of hood body and a central axis ranges from 20° to 60°.

7. The atomizing nozzle of claim 6, wherein an extension of the columnar end ranges from 1 to 6 mm and its diameter ranges from 4 to 8 mm.

8. The atomizing nozzle of claim 1, wherein the upper end of the fluid-guiding cylinder is provided with at least one radially open liquid outflow hole, and an interior of the spray hood is provided with at least one layer of fluid atomization structure composed of a plurality of fan-shaped projections, the fan-shaped projections are evenly distributed along the circumference of the spray holes, and to pressurize the liquid flowing out through the liquid outflow hole to guide it to spiral forward to the spray hole.

9. The atomizing nozzle of claim 8, wherein the fluid atomization structure further comprises a conical channel, the conical channel tapers in cross-section at an end proximate to the spray hole to pressurize the fluid flowing to the spray hole.

10. A nasal drug delivery device, comprising:
an atomizing nozzle, comprising:
a spray hood, comprising a conical hood body and a connecting cylinder provided inside the hood body and connected to an inner top of the hood body, the wall of the connecting cylinder is provided with a bayonet structure or a claw structure for non-detachable snap fit with a corresponding claw structure or a bayonet structure on an outer side of a syringe extending into the connecting cylinder, a columnar end extends from an external tip of the hood body for extending into an internal nasal cavity, the connecting cylinder, the columnar end and a portion of the hood body there between jointly form a communicating channel, a front end of the columnar end is provided with a spray hole, and
a fluid-guiding cylinder, provided in the communicating channel and having an interference fit with an inner wall of the channel, an upper end of the fluid-guiding cylinder is provided with a liquid outflow hole, the fluid in the fluid-guiding cylinder flows out through the liquid outflow hole and is guided to be sprayed out from the spray hole, and
a syringe, an upper end of the syringe is inserted into the lower end of the atomizing nozzle and snap-fit with the atomizing nozzle.

11. The atomizing nozzle of claim 10, wherein the bayonet structure or the claw structure on the wall of the connecting cylinder is located on a lower side of the wall of the connecting cylinder below a bottom horizontal plane of the hood body.

12. The atomizing nozzle of claim 10, wherein an inner lower side edge of the connecting cylinder is provided with at least one locating portion recessed in a direction close to the hood body, the locating portion is in the same longitudinal axis as the bayonet structure or the claw structure on the lower side of the wall of the connecting cylinder to circumferentially locate the bayonet structure or the claw structure on the outer side of syringe extending into the connecting cylinder.

13. The atomizing nozzle of claim 12, wherein a lower edge of the bayonet structure on the lower side of the wall of the connecting cylinder is curved, and tangents are made along two arc endpoints of the lower edge, and the angle between the tangents and the horizontal plane ranges from 0° to 70°.

14. The atomizing nozzle of claim 13, wherein the angle between tangent and horizontal plane is 18°.

15. The atomizing nozzle of claim 10, wherein the angle between a conical surface of hood body and a central axis ranges from 20° to 60°.

16. The atomizing nozzle of claim 15, wherein an extension of the columnar end ranges from 1 to 6 mm and its diameter ranges from 4 to 8 mm.

17. The atomizing nozzle of claim 10, wherein the upper end of the fluid-guiding cylinder is provided with at least one radially open liquid outflow hole, and an interior of the spray hood is provided with at least one layer of fluid atomization structure composed of a plurality of fan-shaped projections, the fan-shaped projections are evenly distributed along the circumference of the spray holes, and to pressurize the liquid flowing out through the liquid outflow hole to guide it to spiral forward to the spray hole.

18. The atomizing nozzle of claim 17, wherein the fluid atomization structure further comprises a conical channel, the conical channel tapers in cross-section at an end proximate to the spray hole to pressurize the fluid flowing to the spray hole.
